(19) United States
(12) Patent Application Publication
(10) Pub. No.: US 2011/0243843 A1
(43) Pub. Date: Oct. 6, 2011

(54) FLUORESCENT SILICA NANOPARTICLES FOR DETECTING LYMPH NODE AND THE IDENTIFICATION METHOD OF LYMPH NODE USING THEREOF

(75) Inventors: DooSoo Chung, Seoul (KR); Keonwook Kang, Seoul (KR); Yonghyun Jeon, Seoul (KR); Younghwa Kim, Seoul (KR); Zeid A. Alothman, Riyadh (SA); Saud I. Airesayes, Riyadh (SA); Kihwan Choi, Seoul (KR); Nawal A. Aiarfaj, Riyadh (SA); Jingyu Piao, Seoul (KR); Salma A. Aitamimi, Riyadh (SA); Bo Quan, Seoul (KR)

(73) Assignees: SNU R&DB Foundation, Gwanak-Gu, Seoul (KR); The Intellectual Propert and Technology Licensing Program, Riyadh (SA)

(21) Appl. No.: 12/595,502

(22) PCT Filed: Sep. 9, 2009
(86) PCT No.: PCT/KR2009/005118

§ 371 Date: Jun. 27, 2011

(30) Foreign Application Priority Data
Sep. 9, 2008 (KR) 10-2008-0089013

(51) Int. Cl. A61K 51/04; C07F 7/02; C07D 311/04; A61K 31/404; C12Q 1/02; A61P 35/00

(52) U.S. Cl. 424/1.65; 548/406; 549/214; 424/9.6; 435/29

(57) ABSTRACT

The present invention relates to fluorescent silica nanoparticles for detecting lymph node and identification method of lymph node using thereof. The functionalized silica nanoparticles containing fluorescent dye of this invention have a promising potential for sentinel node detection in the surgical field through fluorescent imaging.

### TECHNICAL FIELD

This invention relates to fluorescent silica nanoparticles for detecting lymph node and identification method of lymph node using thereof.

### BACKGROUND ART

Sentinel lymph node detection based on the use of radiolabeled colloid nanoparticles combined with blue dye during surgery in early breast cancer has become a standard means of reducing the extent of surgical exploration and post-operative morbidity (Radovanovic Z, Golubovic A, Plzak A, Stojiljkovic B, Radovanovic D., Eur J Surg Oncol 2004; 30:913-7; Rodier J F, Velten M, Wilt M, Martel P, Ferron G, Vaini-Elies V, et al., J Clin Oncol 2007; 25:3664-9). Moreover, sentinel node detection has now been adopted for other types of cancers (Roberts A A, Cochran A J., J Surg Oncol 2004; 85:152-61; Aikou T, Kitagawa Y, Kitajima M, Uenosono Y, Bilchik A J, Martinez S R, et al., Cancer Metastasis Rev 2006; 25:269-77). Although the amount of radioactivity used for sentinel node detection is low and generally considered safe, general concern of using radioisotope has been still aroused in the nursing and pathologic staff (Nejc D, Wrzesien M, Piekarski J, Olszewski J, Pluta P, Kusmierek J, et al., Eur J Surg Oncol 2006; 32:133-8). Accordingly, the uses of various non-radioactive materials, such as, fluorophore dyes and nanoparticles, have been investigated in the context of sentinel node detection (Table 1). However, the low molecular weights of fluorophore dyes mean that their residence times at sentinel nodes are limited, and thus, researchers have been trying to develop new materials for this purpose. Quantum dots (QDs) and macromolecular MRI contrast materials in combination with in vivo imaging systems have been used to locate sentinel lymph nodes in living organisms with high sensitivity and resolution. However, despite their potential benefits, the practical applications of quantum dots are limited by poor bio-compatibility and potential toxicity (Hardman R., Environ Health Perspect 2006; 114:165-72; Zhang T, Stilwell J L, Gerion D, Ding L, Elboudwarej O, Cooke P A, et al., Nano Lett 2006; 6:800-8).

TABLE 1
Studies conducted on sentinel lymph node
detection using nanoparticles and dyes

| Year | Authors | Objects | Used material |
|---|---|---|---|
| 2008 | Sevick- | Human | ICG(Sevick-Muraca EM, Sharma R, |
| | Muraca et al. | | Rasmussen JC, Marshall MV, |
| | | | Wendt JA, Pham HQ, et al., |
| | | | Radiology 2008; 246: 734-41) |
| 2007 | Kobayashi | Nude | Qdot 565, 605, 655, 705, and |
| | et al. | mouse | 800(Kobayashi H, Hama Y, Koyama Y, |
| | | | Barrett T, Regino CA, Urano Y, et al., |
| | | | Nano Lett 2007; 7: 1711-6) |
| 2004 | Kim et al. | Swine | Quantum dot 840(Kim S, Lim YT, |
| | | | Soltesz EG, De Grand AM, Lee J, |
| | | | Nakayama A, et al., Nat |
| | | | Biotechnol 2004; 22: 93-7) |
| 2005 | Pelosi et al. | Human | ^{99m}Tc-labeledalbumin nanocolloid |
| | | | and blue blue dye(Pelosi E, |
| | | | Ala A, Bello M, Douroukas A, |
| | | | Migliaretti G, Berardengo E, et al., |
| | | | Eur J Nucl Med Mol Imaging |
| | | | 2005; 32: 937-42) |
| 2003 | Josephson | Nude | Cy5.5(Josephson L, Mahmood U, Wunder- |
| | et al. | mouse | baldinger P, Tang Y, Weissleder R., Mol |
| | | | Imaging 2003; 2: 18-23) |
| 2001 | Simmons | Human | Methylene blue dye(Simmons RM, Smith |
| | et al. [symbol] | | SM, Osborne MP., Breast J 2001; 7: 181-3) |
| 2000 | Rety | Rat | Superparamagnetic nanoparticle fer- |
| | et al. [symbol] | | umoxtran(Rety F, Clement O, Siauve N, |
| | | | Cuenod CA, Carnot F, Sich M, et al., J |
| | | | Magn Reson Imaging 2000; 12: 734-9) |
| 1996 | Karakousis | Human | Rosaniline dye(Karakousis CP, Velez AF, |
| | et al. | | Spellman JE, Jr., Scarozza J., Eur J Surg |
| | | | Oncol 1996; 22: 271-5) |
| 1993 | Alex and | Cat | ^{99m}Tc sulfur colloid(Alex JC, Krag DN., |
| | Krag | | Surg Oncol 1993; 2: 137-43) |
| [symbol] | Alex | Human | ^{99m}Tc sulfur colloid(Alex JC, Weaver DL, |
| | et al. [symbol] | | Fairbank JT, Rankin BS, Krag DN., |
| | | | Surg Oncol 1993; 2: 303-8) |
| 1980 | Hirsch | Human | Isosulfan blue dye(Hirsch JI., Am J Hosp |
| | et al. [symbol] | | Pharm 1980; 37: 1182-3) |

ICG; Indocyanine Green,
Qdot; Quantum dot,
^{99m}Tc; Technetium-99m

[symbol]

Functionalized silica nanoparticles can be made by incorporating fluorescent dye molecules within the silica matrix, and can be easily conjugated with many other bio-molecules (Yoon T J, Yu K N, Kim E, Kim J S, Kim B G, Yun S H, et al., Small 2006; 2:209-15; Wang J, Liu G, Lin Y., Small 2006; 2:1134-8; Barik T K, Sahu B, Swain V., Parasitol Res 2008; 103:253-8; Yoon T J, Kim J S, Kim B G, Yu K N, Cho M H, Lee J K., Angew Chem Int Ed Engl 2005; 44:1068-71).

Furthermore, Kim et al. investigated the toxicity and tissue distribution of SiO₂ nanoparticles in mice, and found that they had no significant long-term toxicity under the experimental conditions used (Kim J S, Yoon T J, Yu K N, Kim B G, Park S J, Kim H W, et al., Toxicol Sci 2006; 89:338-47). However, although several studies have concluded that functionalized silica nanoparticles can be applied in various biological and medical areas, functionalized silica nanoparticles was not applied to in vivo animal study using optical imaging.

Also, in conventional examination of sentinel lymph node, we can not confirm radioisotope nanoparticle during operation, and dying material was so small that it pass the sentinel lymph node. Also, conventional nano fluorescent quantum dots use the Cadmium (Cd), so, it is hard to apply those to human body.

The inventors of the present invention tried obtaining optical imaging of living organism using toxicity free material, we knew that silica nanoparticle doped with fluorescent dye is harmless to human and is accumulated in lymph node, especially sentinel lymph node, and we complete this invention by confirming these to be used in clinic.

### DISCLOSURE OF INVENTION

### Technical Problem

It is an object of this invention to provide fluorescent silica nanoparticles which are used in detecting in vivo imaging and identification method of lymph node using thereof.

### Technical Solution

In order to achieve the above object, the present invention provides fluorescent silica nanoparticles which are used in detecting in vivo imaging of lymph node.

The present invention also provides detecting method of in vivo imaging and verifying method of lymph nodes using the fluorescent silica nanoparticles

The present invention describes in detail herein.

This invention provides fluorescent silica nanoparticles which are used in detecting in vivo imaging of lymph node. Preferably, the lymph node is sentinel lymph node, and the fluorescent ingredient out of the fluorescent silica nanoparticles is RITC or indocyanine green, but not as limiting the scope thereof. Also, fluorescent silica nanoparticles can comprise radioisotopes additionally. In this case, these nanoparticles can be useful for PET.

The present invention also provides detecting method of imaging using the fluorescent silica nanoparticles. Preferably, said imaging is selected from the group consisting of in vitro imaging, in vivo imaging, bio-distribution tracing, and cancer cell labeling.

And, the present invention provides the mapping method of lymph nodes comprising the steps of: i) injecting fluorescent silica nanoparticles into examination object; and ii) verifying lymph nodes by detecting the fluorescence of said nanoparticles. This method uses the nature that fluorescent silica nanoparticles are accumulated in lymph node, and is applied in both in vivo and in vitro.

And, the present invention provides the verifying method of lymph nodes comprising the steps of: i) injecting fluorescent silica nanoparticles into examination object and detecting the fluorescence; and ii) injecting more fluorescent silica nanoparticles into examination object and detecting the fluorescence, and then comparing said two fluorescence. The fluorescent intensity is proportional to the injected dosage in the lymph node where the nanoparticles are accumulated, so we could verify the lymph node through the change of fluorescence.

And, the present invention is the verifying method of lymph nodes by confirming the accumulation of fluorescent silica nanoparticles in examination object through detecting the fluorescence after injecting fluorescent silica nanoparticles into examination object. This is very useful to confirm the range of removable cell line or the propriety of removed cell line, and we can confirm these in real time. The mapping through the conventional PET or MRI can not provide us with confirmation of the propriety of the mapping, but this invention can confirm it in real time.

Also, the present invention provides the examination method of cell lines comprising the steps of: i) injecting fluorescent silica nanoparticles into cell lines; ii) washing said cell lines; and iii) detecting the fluorescence of said washed cell lines. The conventional cancer checkups are mostly the microscopic tissue checkup or immune chromosome checkup through specific antigen-antibody reaction. But, it is desirable that more concrete checkups are executed when the brief (or rough) checkup is executed in advance and there are some abnormal symptom, for efficiency in time, money and equipment. The present invention will be the useful method to check the abnormality using very simple method and equipment. In other words, the present invention is possible to check the abnormality without specific antibody coupling. Of course, the method of specific antibody coupling can be used together, or full scanning could be applied with PET using the radioactive tagged nano particle.

Silica nanoparticle fluorescent material is applicable in clinic because its influence to the human body is minimal; in addition, full imaging of body is possible when radioisotope for PET is used together.

The present invention can provide sentinel lymph node PET/fluorescent dual imaging in case of using radioisotope labeled fluorescent silica nanoparticle.

In this invention, to evaluate the feasibility of using functionalized silica nanoparticles as imaging probes for intra-operative sentinel node detection, we localized and dissected sentinel nodes in nude mice using RITC functionalized silica (RITC-SiO₂) nanoparticles in vivo by using a fluorescent imaging system and confirmed the presence of rhodamine in sentinel nodes by fluorescence microscopy and biodistribution study.

Rhodamines are stable species and emit at 500˜600 nm in the visible with high quantum yields. Furthermore, the rhodamines are generally non-toxic, and are soluble in water, methanol, and ethanol. This might be possible if a near infrared ray dye, such as, indocyanine green (ICG) were incorporated into the silica matrix.

RITC-SiO₂ nanoparticles were also examined under a transmission electron microscope (TEM). As shown in FIG. 1, the nanoparticles were uniform in size and had a mean diameter of 75±7 nm. Furthermore, after continuous excitation, RITC-SiO₂ nanoparticles showed only slight photobleaching. However, under the same conditions, the fluorescence intensity of pure RITC decreased by 38%. These results indicate that RITC-SiO₂ nanoparticles are more photostable than the free dye (FIG. 2).

Fluorescent silica nanoparticles have much usefulness. It is useful to find sentinel lymph node showing the fluorescence by selective staying. Target coupled material such as fluorescent silica nanoparticles-antibody shows cell specific coupling as fluorescence, so it is useful to checkup the existence of target. For example, we can checkup the cancer through fluorescence when fluorescent silica nanoparticles-Erbitux (cetuximab) can couple with cancer cell having EGFR, and also can be used in prediction of cure effect of cetuximab.

These inventors examined the feasibility of fluorescent imaging for sentinel lymph node detection using rhodamine doped silica nanoparticles. These nanoparticles were injected subcutaneously into the right foot-pads of the fore legs of nude mice. Whole-body images were serially obtained at 5, 10, 15, 20, 30 min after injection using an in vivo imaging system. At 5 min post-injection, fluorescent signals were observed in right axillary lymph nodes (ALN) and at injection sites. Fluorescent signals were also observed at these locations in a bio-distribution study. In addition, fluorescence was detected in frozen ALN sections microscopically. A functionalized silica nanoparticles containing fluorescent dye have a promising potential for sentinel node detection in the surgical field through fluorescent imaging.

### Advantageous Effects

As explained hereinbefore, the functionalized silica nanoparticles containing fluorescent dye of this invention have a promising potential for sentinel node detection in the surgical field through fluorescent imaging.

### BRIEF DESCRIPTION OF DRAWINGS

FIG. 1, 2 is transmission electron micrographs of RITC-SiO₂ nanoparticles (FIG. 1, Photostability experiment in TEM image of RITC-SiO₂ nanoparticles; FIG. 2, Photobleaching profiles of RITC-SiO₂ nanoparticles and rhodamine β isothiocyanate dye (RITC) were obtained using a spectrofluorometer; samples were continuously illuminated and data points were collected every second. The excitation and emission wavelengths were 435 nm and 475 nm, respectively. Fluorescence has been normalized to the same initial intensity.

FIG. 3, 4 is in vitro imaging using IVIS100 (Fluorescent/bioluminescence imaging machine) after allotment nano silica to e-tube variously (FIG. 3), and the quantified graph of said imaging according to the volume (FIG. 4).

FIG. 5, 6 is in vitro fluorescent imaging (FIG. 5) of the mouse after hypodermic injection of silica nanoparticle with various concentrations, and the quantified graph (F 6) of said imaging.

FIG. 7, 8 is in vivo biodistribution (FIG. 7) of nano silica with biooptic imaging equipment in a day after manufactured nano silica was injected into right fore foot pad, and the imaging after extraction of all organs (FIG. 8).

FIG. 9 is in vivo sentinel lymph node imaging: A is in vivo fluorescence imaging of mice injected with RITC-SiO₂ nanoparticles. Mice were injected with RITC-SiO₂ nanoparticles (40 μg/40 μl s.c.) into right fore foot-pads, and fluorescence images were acquired 5 min post-injection. B is in vivo fluorescence images of mice injected with RITC-SiO₂ nanoparticles after stripping skin. Skin was removed and was imaged at 5 min post-injection. C is Ex vivo imaging of axillary lymph nodes. After sacrifice, axillary lymph nodes was extracted and imaged.

FIG. 10 is Ex vivo validation of RITC-SiO₂ nanoparticles. A is Ex vivo fluorescent image of extracted lymph nodes. In vivo fluorescent images were acquired after skin removal at 30 min post RITC-SiO₂ injections to locate sentinel lymph nodes. After in vivo whole body imaging acquisition, mice were sacrificed and eight lymph nodes were extracted to detect specific uptakes in axillary and brachial lymph nodes. B is Ex vivo fluorescence imaging of organs. Animals were sacrificed and all organs were removed and imaged at 30 min post RITC-SiO₂ injection. ALN; axillary lymph node, IN; inguinal lymph node, SN; sciatic lymph node, BLN; brachial lymph node, SCN; superficial cervical lymph node. All images were acquired under the same experimental conditions.

FIG. 11 is biodistribution of ⁶⁸Ga-NOTA-RITC-SiO₂ nanoparticles in nude mice. Mice were sacrificed 30 min after injecting 50 mCi of ⁶⁸Ga-NOTA-RITC-SiO₂ s.c. into the right fore foot-pads. Organs were then removed and weighed, and radioactivities were measured. ALN; axillary lymph node, IN; inguinal lymph node, SN; sciatic lymph node, BLN; brachial lymph node, SCN; superficial cervical lymph node. Data are expressed as % ID/g of tissue. n=5 mice

FIG. 12 is fluorescence microscopic imaging of axillary and brachial lymph nodes sections. A and B is Ex vivo fluorescence imaging of axillary and brachial lymph nodes near footpads injected with RITC-SiO₂ nanoparticles. C and D is Ex vivo fluorescence images of axillary and brachial lymph nodes near footpads injected with PBS. RITC-SiO₂ nanoparticles were injected into right fore foot-pads of nude mice. Axillary and brachial lymph nodes near injected or non-injected foot-pads were excised and frozen sections were prepared to determine the biodistribution of RITC-SiO₂ nanoparticles. All images were acquired using the same experimental conditions and are displayed in the same scale. Scare bar: 5 μm

FIG. 13 is the result analyzed from FACS after treatment nano silica to A431.

FIG. 14 is the imaging from fluorescent microscope after treatment nano silica to A431.

FIG. 15 is the result analyzed from FACS after labeling nano silica-cetuximab to the cell line which is EGFR positive.

FIG. 16 is the imaging from fluorescent microscope after labeling nano silica-cetuximab to the cell line which is EGFR positive.

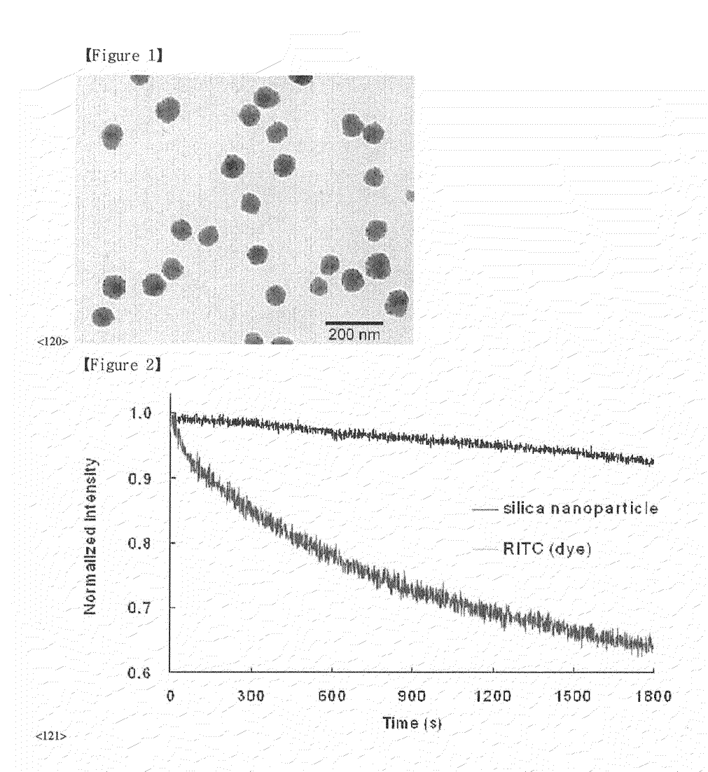

### BEST MODE FOR CARRYING OUT THE INVENTION

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

### Example 1

### Animals and Chemicals

<1-1> Animals

Specific pathogen-free six-week-old female BALB/c nude mice were obtained from SLC Inc. (Japan). All animal experiments were performed after receiving approval from the Institutional Animal Care and Use Committee (IACUC) of the Clinical Research Institute at Seoul National University Hospital. In addition, National Research Council (NRC) guidelines for the care and use of laboratory animals (revised 1996) were observed throughout.

<1-2> Chemicals

Rhodamine B isothiocyanate (RITC), 3-(aminopropyl)triethoxysilane (APTS), and phosphate buffered saline (PBS, pH 7.4) were obtained from Sigma (St. Louis, Mo.). Tetraethyl orthosilicate (TEOS), and 29 wt % aqueous ammonia solution were from Aldrich (Milwaukee, Wis.). 2-[Methoxy(polyethylenoxy)propyl]trimethoxysilane (PEG-silane, 90%) were from Gelest (Morrisville, Pa.).

### Example 2

### Preparation of RITC Functionalized Silica Nanoparticles

ITC-doped silica nanoparticles were synthesized using the Stober method (Wang L, Tan W., Nano Letters 2006; 6:84-8; Stōber W, Fink A, Bohn E., Journal of Colloid and Interface Science 1968; 26:62-9; Smith J E, Wang L, Tan W., Trac-Trends in Analytical Chemistry 2006; 25:848-55; Santra S, Liesenfeld B, Dutta D, Chatel D, Batich C D, Tan W, et al., Journal of Nanoscience and Nanotechnology 2005; 5:899-904). Briefly, after adding 3.08 uL APTS to 1 mL RICT solution (11 uM), the mixture was vigorously stirred for 17 h to link RITC with APTS for the covalent conjugation of RITC to the nanoparticles. Next, 622 uL ammonium hydroxide solution was added to the mixture and the activation reaction was allowed to continue for 5 h at room temperature. After 355 uL TEOS was added, the mixture was stirred for 36 h at room temperature. 5 uL APTS and 5 uL PEG-silane were added to obtain amine-modified RITC-doped silica nanoparticles. After stirring for 24 h at room temperature, the modified silica nanoparticles were isolated from unreacted silica compounds by centrifugation at 14000 rpm for 30 min and washed with ethanol twice and with PBS several times. Final products were redispersed in PBS and stored at 4 C for future use. Size and morphology of nanoparticles was measured by a transmission electron microscope (H-7600, Hitachi, Tokyo, Japan).

In order to investigate the photostability of silica nanoparticles when they are exposed to an aqueous environment for biological applications, the functionalized silica nanoparticles containing rhodamine B isothiocyanate and rhodamine B isothiocyanate (RITC) dye were taken for the photobleaching experiment in aqueous solution excited with a 150 xenon lamp. A 100 uL portion of sample solutions were taken in a quartz cell, and the experiments were conducted on a FP-750 spectrofluorometer (Jasco, Tokyo, Japan).

### Example 3

### Synthesis of ⁶⁸Ga-NOTA-RITC-SiO₂ Nanoparticles

To the sodium carbonate solution (0.2 M, 1 mL, RITC-SiO₂ nanoparticles solution (100 L) and 2-(4′-isothiocyanatobenzyl)-1,4,7-triazacyclononane-1,4,7-triacetic acid (p-SCN-Bn-NOTA, 2.0 mg, 3.6 nmol) were added. The mixture was stirred for 12 h at room temperature and centrifuged to remove supernatant, and washed with ethanol (1 mL) and water (1 mL), successively. The orange colored precipitate was re-dispersed in water (1 mL) and kept at −20 C. RITC-SiO₂ nanoparticles-SCN-NOTA solution (100 L) and ⁶⁸GaCl₃ solution (287 MBq, 900 L), which was freshly eluted from ⁶⁸Ge—⁶⁸Ga generator, were mixed and sodium phosphate solution (0.5 M, 220 L) was added to adjust pH 5. The mixture was mixed and kept at 90° C. for 20 min. After the reaction, the reaction mixture was centrifuged and washed with de-ionized water (1 mL), and the precipitate was re-dispersed in water (1 mL) before injection. The radiochemical yield and radiochemical purity were checked by ITLC-SG (eluent: 0.1 M sodium carbonate or 0.1 M citric acid solution). The R_{f} value of ⁶⁸Ga-NOTA-SiO₂ nanoparticles was 0.1 with both eluents, and that of free ⁶⁸Ga was 0.1 using 0.1 M sodium carbonate solution and 1.0 using 0.1 M citric acid solution. The radiochemical yield was over 95% and radiochemical purity was over 99% after the purification.

### Example 4

### In Vivo Fluorescence Imaging Study

Fluorescence images were obtained using a Maestro In Vivo Imaging System (CRi Inc., Woburn, Mass.) for data acquisition and analysis. Before imaging, mice were anesthetized i.p. with a solution containing 8 mg/mL ketamine (Ketalar, Panpharma, Fougres, France) and 0.8 mg/mL xylazine (Rompun, Bayer Pharma, Puteaux, France) at 0.01 mL/g of body weight. RITC-SiO₂ nanoparticles (40 ug/40 uL) were injected s.c. into the right fore foot-pads of nude mice. Fluorescence measurements were performed at 5 min after foot-pad injections. In vivo fluorescence Measurements were taken on top of ALNs (axillary lymph nodes) after skin removal.

In all cases, optical image sets were acquired using a green filter set (a band-pass filter from 503 to 555 nm and a long-pass filter of 580 nm. which were used for excitation and emission, respectively) to acquire one complete image cube. The tunable filter was automatically increased in 10-nm increments from 550 to 800 nm. A camera was used to capture images at each wavelength using a constant exposure.

<4-1> In Vitro Imaging of Fluorescent Silica Nanoparticles

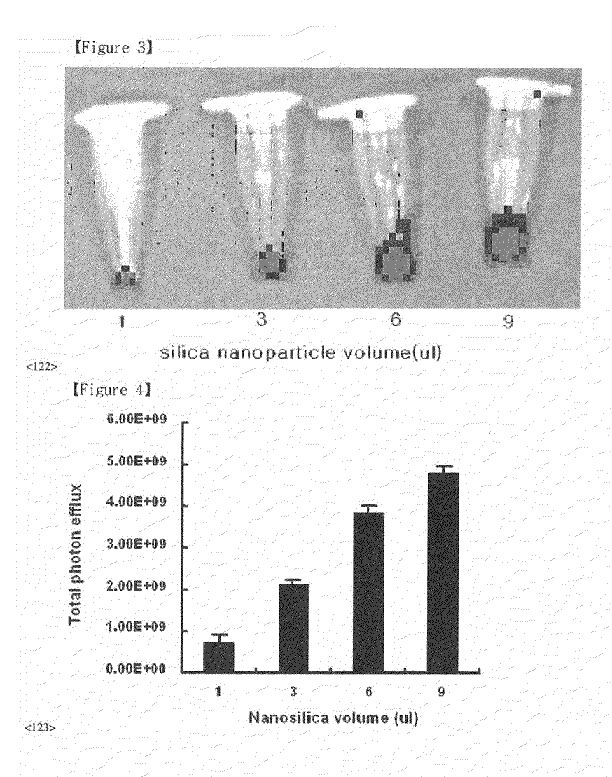

We get the in vitro imaging using IVIS100 (Fluorescent/bioluminescence imaging machine) after allotment to e-tube (1.5 ml) of 1, 3, 6, 9 μl nano silica with fluorescent dye respectively (FIG. 3), and the quantified graph of said imaging (FIG. 4). It is confirmed that fluorescent imaging increases in proportion to volume of silica nanoparticle.

<4-2> In Vivo Imaging of Fluorescent Silica Nanoparticles

We get the in vivo imaging after subcutaneous injection of fluorescent silica nanoparticle into dorsal region. We get the full imaging using IVIS100 (Fluorescent/bioluminescence imaging machine) after injecting fluorescent silica nanoparticle into footpad.

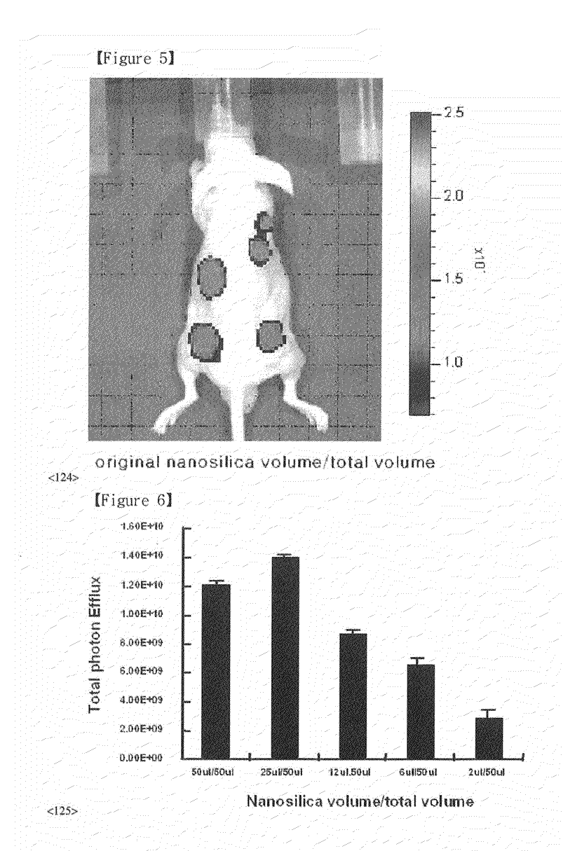

In the concrete, We get the full imaging using IVIS100 after subcutaneous injection of silica nanoparticle 2, 6, 12, 25, 50 μg/50 μl in PBS (FIG. 5). Quantitative analysis is carried out using the imaging around injection area after getting full imaging (FIG. 6). It is confirmed that imaging in injection area increases in proportion to volume of silica nanoparticle.

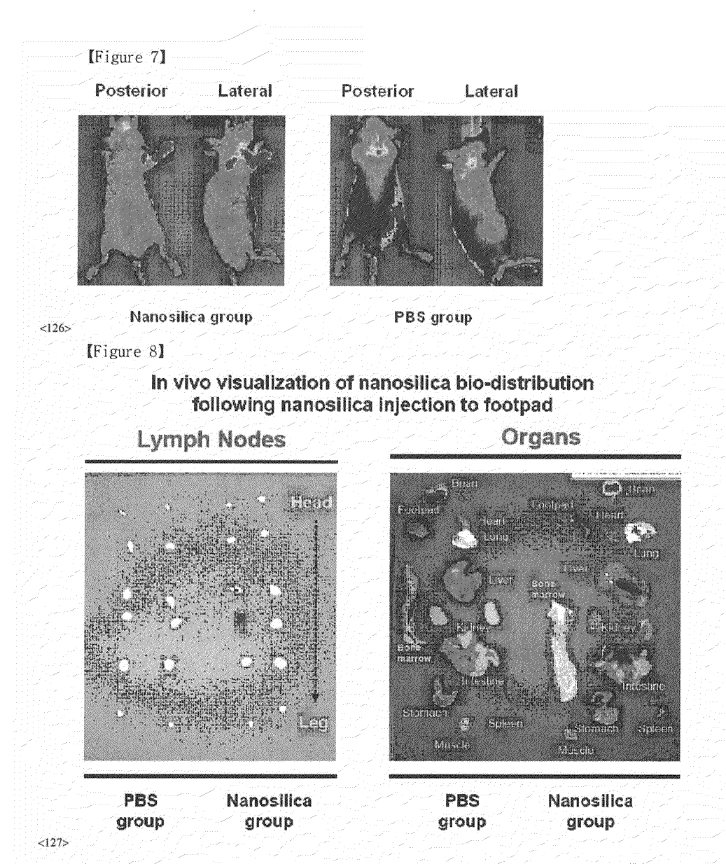

Also, we get the in vivo imaging after subcutaneous injection of fluorescent silica nanoparticle into footpad. In the concrete, we photograph distribution of silica nanoparticle using IVIS100 after injection of silica nanoparticle 50 μl into footpad (FIG. 7). Also, we photograph after the removal of organs (FIG. 8). As a result, we get the strong fluorescent imaging from injection area of footpad, and get the fluorescent imaging from draining lymph nodes.

### Example 5

### Biodistribution Chasing of Fluorescent Silica Nanoparticles

We trace bio-distribution of fluorescent silica nanoparticles after injection of RITC-SiO₂ nanoparticles. In particular, mice (n=5) were sacrificed 30 min after injecting RITC-SiO₂ nanoparticles (40 ug/40 ul) to right fore foot-pads of immunocompetent Balb/c mice. Organs, including lymph nodes, were removed and imaged using a Maestro imaging system. As a result, we can observe the nano particles accumulated in draining lymph nodes, liver, kidney, stomach, and bone marrow, at 3 hours and 24 hours later after injection.

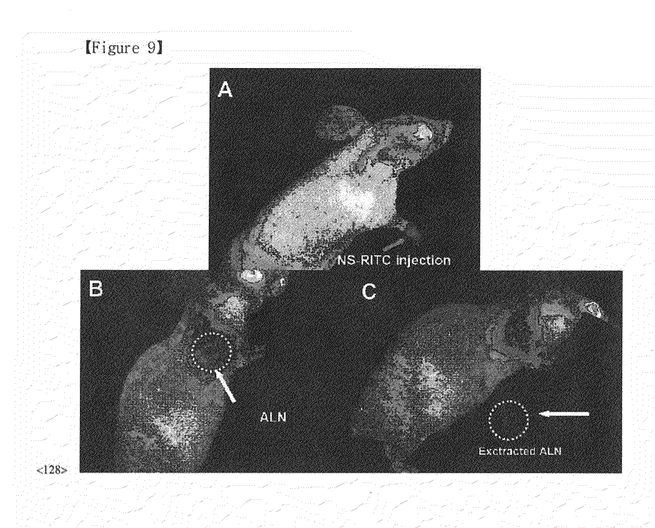

Also, the inventors proved that functionalized silica nanoparticles move to ALN (axillary lymph nodes) near right foot pad on the fore leg. Fluorescent signals were observed at injection sites but not at axillary lymph nodes (ALN) in intact skin 5 min after injecting silica nanoparticles into right footpads on the fore leg (FIG. 9 A). To detect fluorescent signals in ALNs, surrounding skin was removed before image acquisition. Drainage toward ALNs can be observed in the stripped region in living mice (FIGS. 9 B and C).

### Example 6

### Biodistribution Study Using ⁶⁸Ga-NOTA-RITC-SiO₂ Nanoparticles

We study Biodistribution of nanoparticle using ⁶⁸Ga-NOTA-RITC-SiO₂ nanoparticles in the example 3. Mice (n=5) were sacrificed and inspected distribution of internal organs in vivo, 30 min after administering ⁶⁸Ga-NOTA-RITC-SiO₂ (50 mCi/50 ul) to right fore foot-pads of immunocompetent Balb/c mice. Organs were removed, weighed, and counted for radioactivity using a gamma counter. Results are expressed as percentages of doses injected per gram of tissue (% ID/g). As a result, it is confirmed that plenty of radioisotope are absorbed around draining lymph nodes.

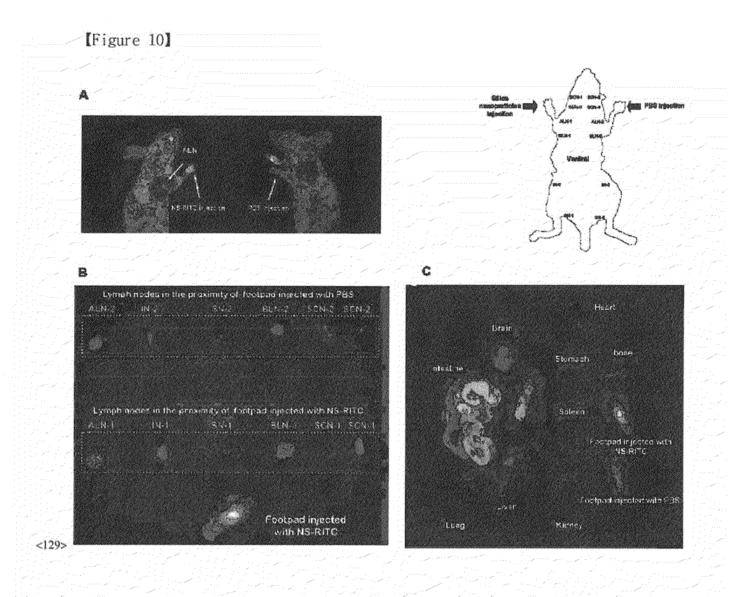

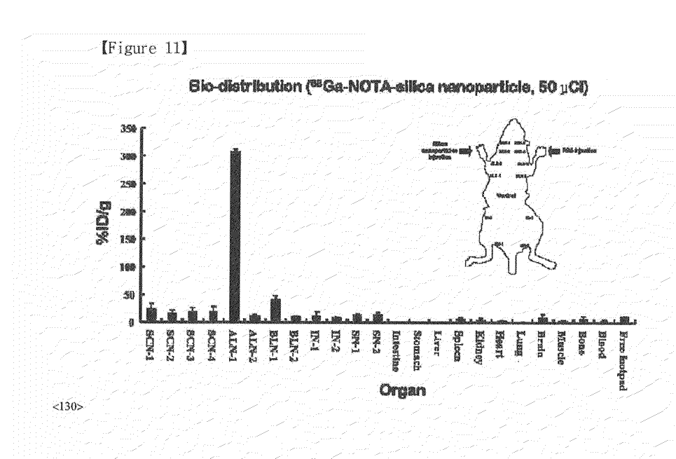

Mice were injected with silica nanoparticles and sacrificed 30 min post-injection. All organs including lymph nodes were removed and imaged. Except for three organs (axillary lymph node, brachial lymph node, and injection foot-pad), fluorescence signals were not detected in the other tested organs (FIG. 10). Also, we examined bio-distribution of ⁶⁸Ga-NOTA-RITC-SiO₂ in nude mouse. The % ID/g of axillary lymph node, brachial lymph node aroud foot-pad treated with ⁶⁸Ga-NOTA-RITC-SiO₂ nanoparticle is respectively 308.3 3.4 and 41.5 6.1 (FIG. 11). The radioactivity of ⁶⁸Ga is not found in any other organs significantly (for example in liver, lungs, brain, spleen and kidney). FIG. 10 and FIG. 11 prove that the bio-distributions of RITC-SiO₂ and ⁶⁸Ga-NOTA-RITC-SiO₂ are similar.

### Example 7

### Fluorescence Microscopy

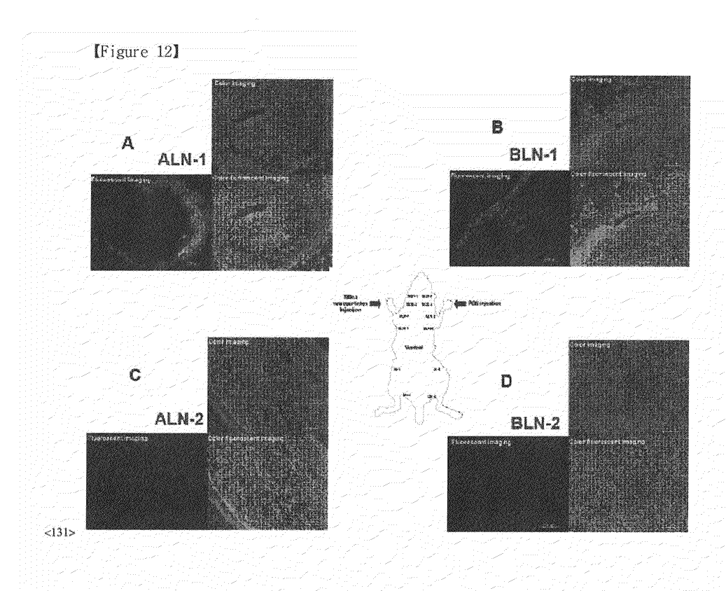

After sacrificing mice, lymph nodes (axillary lymph nodes and brachial lymph nodes) were removed and frozen at −80 C. Frozen sections (30) of all lymph nodes were prepared after embedding in Tissue Tek O.C.T. compound (Sakura Finetek, Torrance, Calif., USA). Fluorescence was observed under an upright epifluorescence microscope (IX-71 Provis, Olympus, Rungis, France) equipped with a 100 W mercury vapor lamp and a Peltier cooled CCD camera (DP71, Olympus) (FIG. 12). The filter set used consisted of a 400-440 nm band pass excitation filter, a 570 nm dichromic mirror, and a 590 nm long pass filter. Fluorescence images were recorded at a magnification of ×40.

Fluorescence microscopy demonstrated that RITC-SiO₂ nanoparticles accumulated in the trabecular and medullary sinuses of axillary and brachial lymph nodes near injected footpads at 30 min post-injection.

### Example 8

### In Vitro Cell Labeling Using Fluorescent Silica Nanoparticles

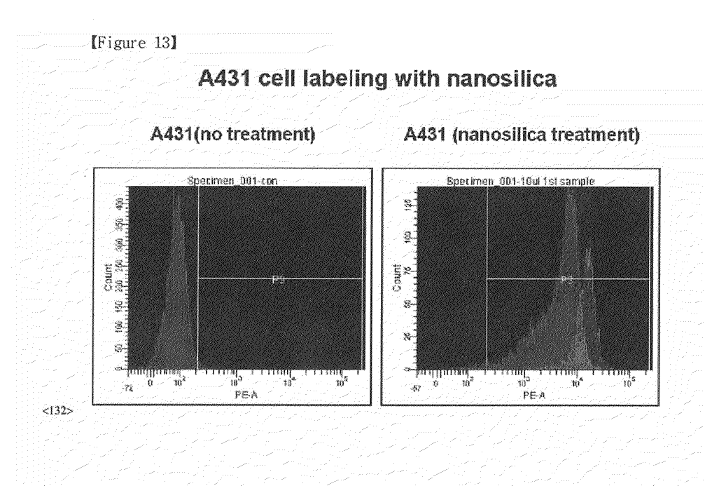

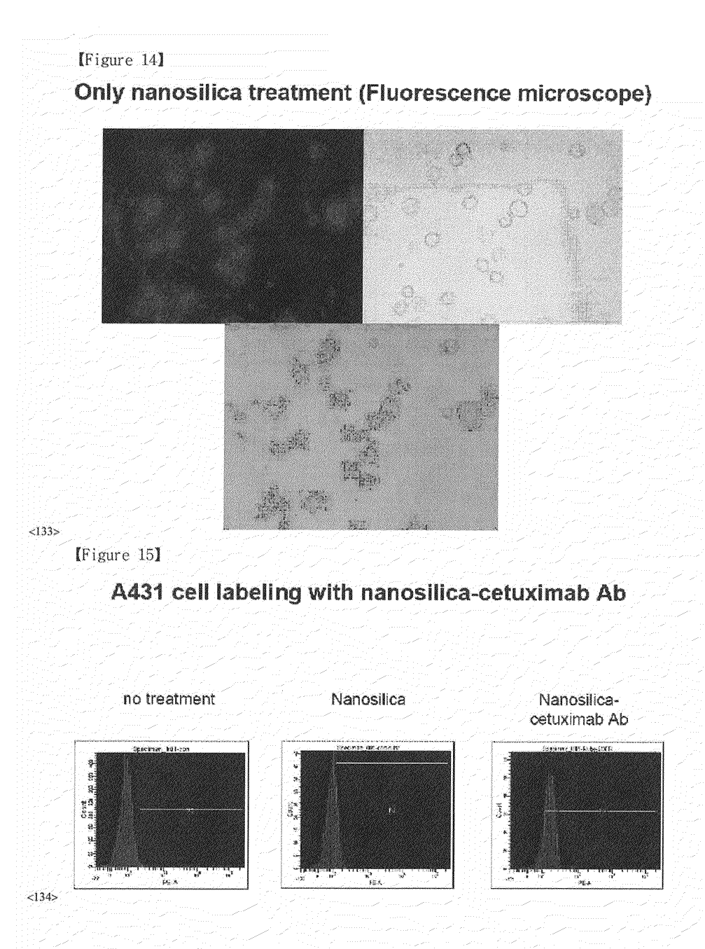

We allot 5×10⁵ A431 (human epithelial carcinoma cell line) to the FACS tube. We washed cell with FACS buffer (0.1% BSA in PBS) after concentration of cell line using centrifuge. We keep it in ice for 20 min after injection of 10 μl nano silica to prepared cell line. We washed labeled cancer cell line twice with FACS buffer. We confirm the extent of labeling with flow cytometry and fluorescent microscope (FIGS. 13 and 14). As a result, all A431 cancer cell line is labeled through flow cytometry analysis, and labeling is at inner cell or cell surface through fluorescent microscope analysis.

### Example 9

### In Vitro Imaging Using Fluorescent Silica Nanoparticles and Cetuximab

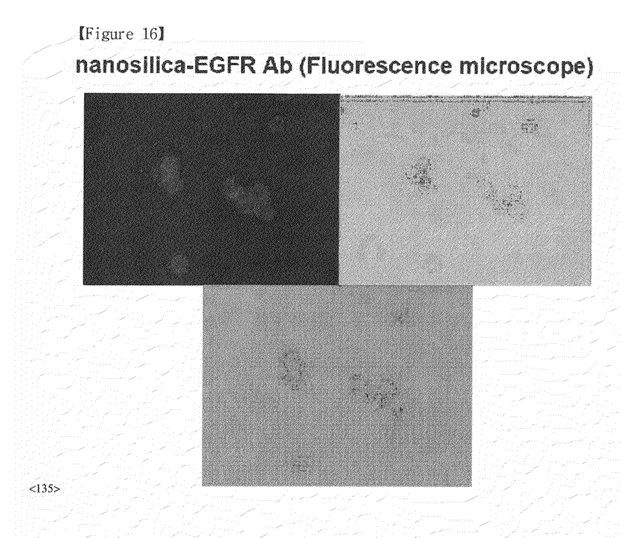

We labeled A431 cell line which is EGFR positive cell with it after connecting fluorescent silica nanoparticles and cetuximab (EGFR targeting antibody), and get the in vitro imaging. We confirm the extent of labeling of cancer cell line with flow cytometry and fluorescent microscope after labeling 5×10⁵ A431 using nano silica-EGFR Ab (FIGS. 15 and 16). As a result, all A431 cancer cell line is labeled through flow cytometry analysis, and we confirm specific coupling at cell surface through fluorescent microscope analysis.

The above results suggest the following; 1) RITC-SiO₂ nanoparticles were small enough to travel freely through lymphatic channels, but are trapped in lymph nodes, and 2) that RITC-SiO₂ nanoparticles are suitable for mapping sentinel lymph nodes in surgical fields.

Although functionalized RITC-SiO₂ nanoparticles offer many advantages, further studies are required in clinical models. The RITC-SiO₂ nanoparticles examined in the present study, had a low signal to background ratio, and thus, it was not possible to detect draining lymph nodes in deep tissues. This might be possible if a near infrared ray dye, such as, indocyanine green (ICG) were incorporated into the silica matrix.

To our knowledge, the present invention demonstrates for the first time the delineation of sentinel lymph nodes using non-toxic fluorescent silica nanoparticles in living mice. We conclude that functionalized RITC-SiO₂ nanoparticles have great potential for visualizing sentinel nodes peri-operatively.

1. Fluorescent silica nanoparticles which are used in detecting in vivo imaging of lymph node.

2. Fluorescent silica nanoparticles of claim 1 wherein the lymph node is sentinel lymph node.

3. Fluorescent silica nanoparticles of claim 1 wherein the fluorescent ingredient is RITC or indocyanine green.

4. Fluorescent silica nanoparticles of claim 1, wherein said nanoparticles additionally comprise radioisotopes.

5. Detecting method of imaging using the fluorescent silica nanoparticles according to any one of claim 1 to claim 4.

6. Detecting method of claim 5, wherein said imaging is selected from the group consisting of in vitro imaging, in vivo imaging, bio-distribution tracing, and cancer cell labeling.

7. The mapping method of lymph nodes comprising the steps of:
i) injecting fluorescent silica nanoparticles into examination object; and
ii) verifying lymph nodes by detecting the fluorescence of said nanoparticles.

8. The verifying method of lymph nodes comprising the steps of:
i) injecting fluorescent silica nanoparticles into examination object and detecting the fluorescence; and
ii) injecting more fluorescent silica nanoparticles into examination object and detecting the fluorescence, and then comparing said two fluorescence.

9. The verifying method of lymph nodes by confirming the accumulation of fluorescent silica nanoparticles in examination object through detecting the fluorescence after injecting fluorescent silica nanoparticles into examination object.

10. The examination method of cell lines comprising the steps of:
i) injecting fluorescent silica nanoparticles into cell lines;
ii) washing said cell lines; and
iii) detecting the fluorescence of said washed cell lines.
